(19) United States
(12) Patent Application Publication
(10) Pub. No.: US 2025/0099317 A1
(43) Pub. Date: Mar. 27, 2025

(54) KNEE EXOSKELETON DEVICE

(71) Applicant: THE HONG KONG POLYTECHNIC UNIVERSITY, Hong Kong (CN)

(72) Inventors: Ho Lam HEUNG, Hong Kong (CN); Sheung Mei Shamay NG, Hong Kong (CN); Wai Lung Thomson WONG, Hong Kong (CN)

(21) Appl. No.: 18/725,183

(22) PCT Filed: Dec. 22, 2022
(86) PCT No.: PCT/CN2022/140912

§ 371 Date: Jun. 28, 2024

Related U.S. Application Data
(60) Provisional application No. 63/266,409, filed on Jan. 5, 2022.

(51) Int. Cl. A61H 1/02

(52) U.S. Cl. CPC A61H 1/0214; A61H 1/024; A61H 2201/1238

(57) ABSTRACT

A rotary motion generator for helping a patient to relearn walking is disclosed. The knee exoskeleton device includes a rotary shaft, a moveable lever, and a first elastomeric structure. The rotary shaft is switchable between a one-way rotation mode and a two-way rotation mode in a sagittal plane, and locking the joint position by the one-way rotation mode. The moveable lever protrudes from an outer periphery of the rotary shaft perpendicularly for rotating the rotary shaft. The first elastomeric structure is pneumatically controlled for engaging the moveable lever for generating an output torque and producing the relative rotatory movement of a lower arm with respect to an upper arm. The rotary shaft further comprises a pawl, a second elastomeric structure for projecting or retracting the pawl, and a rotation locking mechanism that switches the rotary shaft between the one-way rotation mode and the two-way rotation mode.

### CROSS-REFERENCE TO RELATED APPLICATIONS

This application claims the benefit of U.S. Provisional Patent Application No. 63/266,409, filed on 5 Jan. 2022, which is incorporated by reference herein in its entirety.

### FIELD OF THE INVENTION

The present invention is generally related to a knee exoskeleton device. Particularly, the present disclosure is related to a pneumatically driven knee exoskeleton device having an electrical stimulation system. The knee exoskeleton device is intended to help a patient, particularly a stroke survivor, to relearn walking by themselves.

### BACKGROUND OF THE INVENTION

Human legs play an important role during normal gait, including shock absorption, stance stabilization, energy conservation, and movement motion. However, mobility and flexibility may deteriorate for the elderly. This problem is particularly significant for patients suffering from a stroke or other terminal diseases. The aging population poses a major challenge to the health care system and the social service, as more countries struggler to support the rising number of elderlies with deteriorated limb mobility or joint flexibility.

One of the medical conditions affecting the independency of the elderly and stroke survivors is the impaired ability to independent walking, which is highly correlated to the health-related quality of life (QOL). The elderly and stroke survivors may not be able to walk for long distances and have gait deficiency. Therefore, there is a tenancy that stroke survivors become inactive after a stroke. Also, the impaired ability to walk directly causes other problems to health, such as muscle weakness, joint contractures, osteoporosis, pressure ulcer, and obesity. Thus, gait rehabilitation is usually the major priority after a stroke.

Hyperextension of the knee is a medical symptom in which an abnormal extension of the knee beyond its neutral position. This is commonly observed in the walking pattern of patients with gait deficiency. This may increase energy expenditure, reduce the ability to balance, and lead to pain, capsular and ligamentous laxity, or bone deformity.

Gait recovery can be accomplished by different rehabilitation interventions. The goal of the interventions is to facilitate a normal walking gait pattern to the paretic leg. For the knee, this generally requires a sufficient contraction of the quadriceps in the stance phase for knee extension and a powerful contraction of the hamstring in the swing phase. An effective gait recovery treatment should incorporate these specific requirements to prevent hyperextension of the knee during walking.

Traditional approaches taken in clinics involve orthotics, occupational therapy (OT) and physiotherapy (PT). The patient may be prescribed with a lightweight knee orthosis that can immobilize the knee into the neutral extended position, which can facilitate motor function and slow down the progression of impaired mobility. However, the patient tends to adapt to compensatory strategies instead in order to walk conveniently, which would promote the development of a wrong gait pattern even though they can regain the ability to walk.

Potential solutions for effective gait rehabilitation have been seen in the field of wearable robotics. These robotic devices are designed to provide direct assistance with knee joint movement and support to body weight. Examples of robotic devices for rehabilitation of the knee joint include U.S. Pat. No. 10,390,973B2 “Interactive exoskeleton robotic knee system”, U.S. Pat. No. 10,682,249B2 “Controllable passive artificial knee”, U.S. Pat. No. 10,238,522B2 “Exoskeleton device and method of impeding relative movement in the exoskeleton device”, U.S. patent application no. US20190192372A1 “Exoskeleton and mounting arrangement”, C.N. patent 109648546B “Adjustable light and thin exoskeleton knee joint driver”, C.N. Pat. No. 111920651B “Exoskeleton joint self-locking mechanism, knee joint and bionic rehabilitation robot”, and C.N. patent application no. 111658434A “Knee hyperextension flexible exoskeleton rehabilitation robot based on pneumatic muscles and rehabilitation method”. These knee exoskeleton robots are mostly constructed with powerful electric motors and rigid metal frameworks, which are still bulky, hard to further reduce the weight, and can pose dangers to patients due to being overpowered. These devices are also mostly developed for laboratory settings. They are tethered to giant power sources, and therefore they become cumbersome and unattractive to be used outside the clinic or laboratory. Particularly, the devices consist of no muscle stimulation ability to maximize patients' voluntary efforts, meaning that human intention is seldom involved in operating the devices and active human-robot interaction is lacking. Therefore, the knee exoskeleton robots of the prior arts have issues associated with effectiveness, complexity, speed of operation, and safety.

Accordingly, there is a need in the art to have a knee exoskeleton device as a daily assistive device that can help the patient to relearn walking by themselves and prevent hyperextension of the knee during walking. Furthermore, other desirable features and characteristics will become apparent from the subsequent detailed description and the appended claims, taken in conjunction with the accompanying drawings and this background of the disclosure.

### SUMMARY OF THE INVENTION

Provided herein is a knee exoskeleton device for preventing hyperextension of the knee during walking and stimulating the selected muscles on the lower limb of the patients. It is an objective of the present disclosure to help a patient, particularly a stroke survivor, to relearn walking by themselves.

According to the first aspect of the present disclosure, a rotary motion generator for use in a robotic device is disclosed. The rotary motion generator is pneumatically driven to cause a relative rotatory movement of a lower arm with respect to an upper arm about an axis parallel to or coincide with a rotation axis of a joint for assisting a joint extension, a joint flexion and capable of locking a joint position. The rotary motion generator includes a rotary shaft switchable between a one-way rotation mode and a two-way rotation mode in a sagittal plane, and locking the joint position by the one-way rotation mode; a moveable lever protruding from an outer periphery of the rotary shaft perpendicularly for rotating the rotary shaft; and a first elastomeric structure pneumatically controlled for engaging the moveable lever to generate an output torque and produce the relative rotatory movement of the lower arm. The rotary shaft further comprises at least a pawl, a second elastomeric structure, and a rotation locking mechanism. The rotation locking mechanism switches the rotary shaft between the one-way rotation mode and the two-way rotation mode by pneumatically controlling the second elastomeric structure to project or retract the pawl.

According to a further embodiment of the present disclosure, the rotation locking mechanism further comprises a plurality of ratchet teeth. The pawl restricts a bidirectional movement of the rotary shaft when engages with the plurality of ratchet teeth.

Preferably, the plurality of ratchet teeth are circumferentially arranged on an inner periphery of the rotary shaft.

According to a further embodiment of the present disclosure, the second elastomeric structure has a bellow tube structure that moves the pawl between an engaged position and a disengaged position. The rotary shaft is prevented from rotating about the axis in an anticlockwise direction when the pawl is in the engaged position. The rotary shaft is allowed to rotate bidirectionally about the axis when the pawl is in the disengaged position.

According to a further embodiment of the present disclosure, the plurality of ratchet teeth comprises a blocking portion and a sloped portion. The pawl engages the plurality of ratchet teeth by abutting the pawl against any one of the plurality ratchet teeth in the engaged position. The blocking portion limits the rotary shaft from rotating in the anticlockwise direction.

Preferably, when the pawl is in the engaged position, the pawl is slidable along the sloped portion so that the rotary shaft can rotate in the clockwise direction to an adjacent ratchet tooth.

According to a further embodiment of the present disclosure, the rotary shaft further comprises an inner housing and a hollowed body. The inner housing is concentrically arranged with respect to the rotary shaft and within the hollowed body, and is not rotated when the rotary shaft rotates. When the pawl is at the engaged position, the pawl is projected from the inner housing by the second elastomeric structure to pivot between the inner housing and the plurality of ratchet teeth for restricting the rotary shaft from rotating about the axis in the anticlockwise direction. When the pawl is at the disengaged position, the pawl is retracted into or partially into the inner housing by the second elastomeric structure to allow the bidirectional movement of the rotary shaft.

According to a further embodiment of the present disclosure, the rotary motion generator includes an outer casing and a radial extension extended radially from the outer casing to the rotary shaft. The first elastomeric structure is circumferentially arranged within the outer casing, and comprises a first end fixed at the radial extension and a second end engaged with the moveable lever.

Preferably, the radial extension is affixed to or formed as an integral part of the outer casing.

Preferably, the outer casing is a hollowed cylinder shell defining an internal space between an inner wall of the outer casing and the rotary shaft, wherein the first elastomeric structure is circumferentially arranged within the outer casing at least partially occupying the internal space.

According to a further embodiment of the present disclosure, the first elastomeric structure comprises a bellow tube structure formed by a plurality of outer convolutions and a plurality of inner convolutions. The plurality of outer convolutions and the plurality of outer convolutions are defined by alternating crests and troughs. The plurality of outer convolutions and the plurality of inner convolutions allow compression or expansion of the first elastomeric structure based on a pressure change supplied to an undulated cavity of the first elastomeric structure.

Preferably, the bellow tube structure has V-shaped convolutions or U-shaped convolutions.

Preferably, the first elastomeric structure comprises a structural mesh connecting the plurality of outer convolutions and the plurality of inner convolutions for supporting the first elastomeric structure and preventing an expansion of the first elastomeric structure in an axial direction.

Preferably, the structural mesh is formed using supporting lengthwise filaments, supporting widthwise filaments, or a patterned mesh selected from a net mesh or a double helical mesh.

Preferably, the structural mesh is impregnated within the first elastomeric structure or mounted to a surface of the first elastomeric structure.

Preferably, the output torque generated is in a range of 10 N-m to 50 N-m; and the rotary motion generator is configured to maintain a range-of-motion of the joint from 60° to 150°.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects and advantages of the present invention are disclosed as illustrated by the embodiments hereinafter.

### BRIEF DESCRIPTION OF THE DRAWINGS

The appended drawings contain figures to further illustrate and clarify the above and other aspects, advantages, and features of the present disclosure. It will be appreciated that these drawings depict only certain embodiments of the present disclosure and are not intended to limit its scope. It will also be appreciated that these drawings are illustrated for simplicity and clarity and have not necessarily been depicted to scale. The present disclosure will now be described and explained with additional specificity and detail through the use of the accompanying drawings in which:

FIG. 1 shows a perspective view of the knee exoskeleton device without the electrical stimulation system in accordance with certain embodiments of the present disclosure;

FIG. 2A shows a side view of the rotary motion generator of the knee exoskeleton device when the rotation locking mechanism is engaged;

FIG. 2B shows a side view of the rotary motion generator of the knee exoskeleton device when the rotation locking mechanism is disengaged;

FIG. 2C shows a side view of the rotary motion generator when the rotary shaft is rotated in an anticlockwise direction;

FIG. 2D shows a side view of the rotary motion generator when the rotary shaft is rotated in a clockwise direction;

FIG. 2E shows a cross-sectional view of the rotary motion generator;

FIG. 3 shows the actuation of the knee exoskeleton device of FIG. 1;

FIG. 4 shows the sensor system of the knee exoskeleton device of FIG. 1;

FIG. 5 is a perspective view of the knee exoskeleton device showing the electrical stimulation system and the supply of pressurized fluid in accordance with certain embodiments of the present disclosure;

FIG. 6 is a schematic diagram of the electrical stimulation system in accordance with certain embodiments of the present disclosure;

FIG. 7 is a table illustrating an example method for determining muscle stimulation and for controlling the knee exoskeleton device at different gait events in accordance with certain embodiments of the present disclosure; and

FIG. 8 is a block diagram of an exemplary knee exoskeleton device in accordance with certain embodiments of the present disclosure.

### DETAILED DESCRIPTION OF THE INVENTION

The following detailed description is merely exemplary in nature and is not intended to limit the disclosure or its application and/or uses. It should be appreciated that a vast number of variations exist. The detailed description will enable those of ordinary skilled in the art to implement an exemplary embodiment of the present disclosure without undue experimentation, and it is understood that various changes or modifications may be made in the function and structure described in the exemplary embodiment without departing from the scope of the present disclosure as set forth in the appended claims.

The benefits, advantages, solutions to problems, and any element(s) that may cause any benefit, advantage, or solution to occur or become more pronounced are not to be construed as a critical, required, or essential features or elements of any or all of the claims. The invention is defined solely by the appended claims including any amendments made during the pendency of this application and all equivalents of those claims as issued.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The terms “comprising,” “having,” and “including” or any other variation thereof, are to be construed as open-ended terms (i.e., meaning “including, but not limited to,”) unless otherwise noted. The use of any and all examples, or exemplary language (e.g., “such as”) provided herein, is intended merely to illuminate the invention better and does not pose a limitation on the scope of the invention unless otherwise claimed. No language in the specification should be construed as indicating any non-claimed element as essential to the practice of the invention. Further, unless expressly stated to the contrary, “or” refers to an inclusive “or” and not to an exclusive “or”. For example, a condition A or B is satisfied by any one of the following: A is true and B is false, A is false and B is true, and both A and B are true. Terms of approximation, such as “about”, “generally”, “approximately”, and “substantially” include values within ten percent greater or less than the stated value.

Unless otherwise defined, all terms (including technical and scientific terms) used in the embodiments of the present invention have the same meaning as commonly understood by an ordinary skilled person in the art to which the present invention belongs.

The term “exoskeleton”, as used herein, refers to a mechanical structure with elements coupled in series, which is adapted to be attached to a user's body.

Embodiments of the present invention include instruments, systems, techniques, and methods for assisted lower limb movement. More specifically, the present disclosure may be applied to, but not limited to, robotic knees, including exoskeleton type robotic knees. Embodiments can include knee exoskeleton devices or robotic knee devices having rotary motion generators with electrical stimulation components. In certain embodiments, the present disclosure can be applied in the medical field or for physiotherapy training. In particular, the device disclosed herein helps a patient to relearn walking after stroke, cerebral ischemia, or other cardiovascular diseases, or helps a patient with lower limb gait deficiencies, including, but not limited to, trauma, incomplete spinal cord injuries, multiple sclerosis, muscular dystrophies, or cerebral palsy to walk. Therefore, the purpose of the present disclosure is to provide a device that can be used by the patient for recovering the knee function.

The present disclosure provides a knee exoskeleton device that combines the features of soft robotics into a mechanical structure for facilitating the movement of the lower limb. The movement can be adaptive to human gait patterns. In certain embodiments, the knee exoskeleton device includes an electrical stimulation system for stimulating muscles in a lower limb constantly to strengthen muscle contraction during gait.

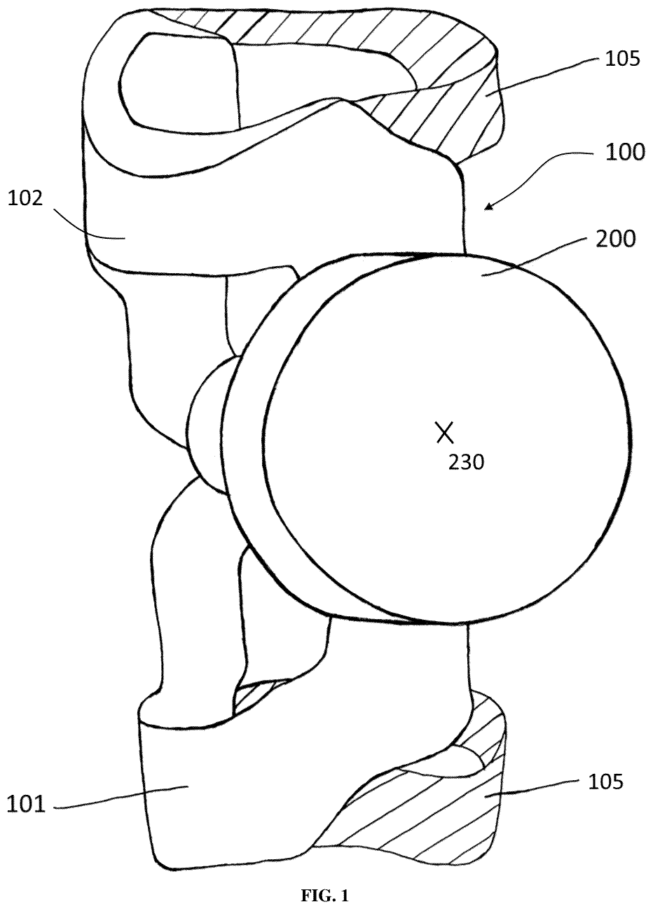

FIG. 1 shows a knee exoskeleton device 100 (without the electrical stimulation system 700) in accordance with a first embodiment of the present disclosure. The knee exoskeleton device 100 may have a lightweight and compact structure, which can be mounted on a user's impaired lower limb. The user may wear the knee exoskeleton device 100 like a normal knee sleeve rather than a bulky lab-based device. Therefore, the knee exoskeleton device 100 may be used as a daily assistive device (instead of a rehabilitation device) helping a patient to relearn walking by themselves after the training for facilitating the recovery of knee function of the patient.

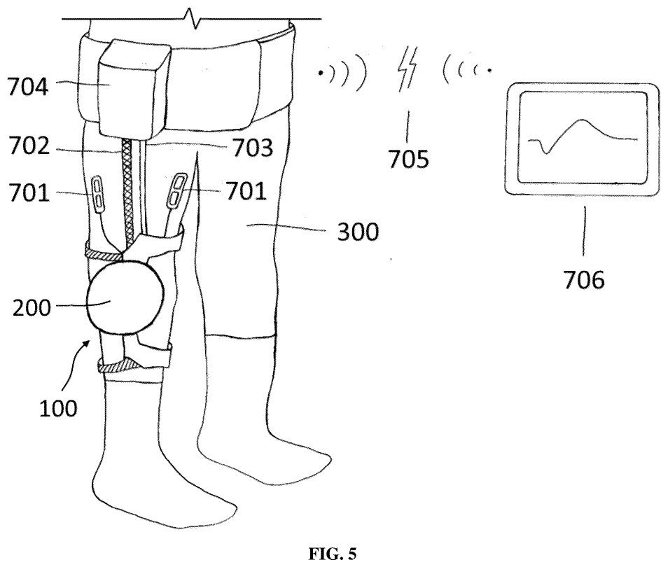

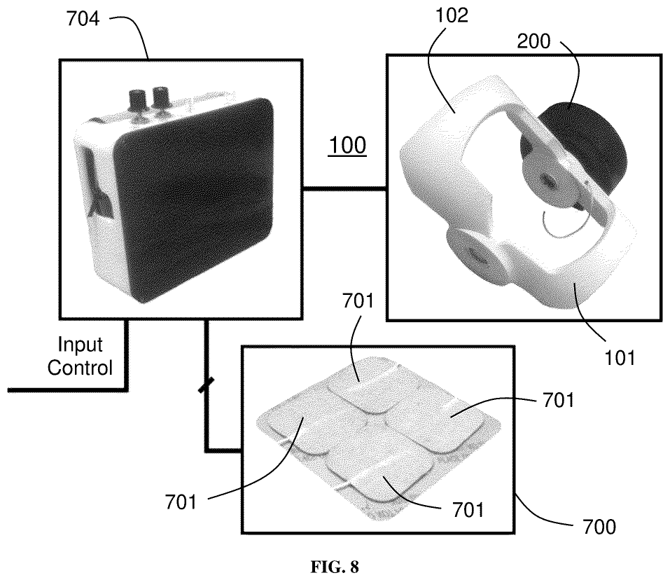

In the illustrated embodiment, the knee exoskeleton device 100 is a robotic device comprising a knee brace having a lower arm 101 and an upper arm 102, which are pivotally coupled to each other at or proximal to a knee joint, and a rotary motion generator 200 coupled to the lower arm 101 and the upper arm 102 of the knee brace for permitting rotation of the lower arm 101 with respect to the upper arm 102. The knee exoskeleton device 100 may further comprise an electrical stimulation system 700 (shown in FIG. 5 and FIG. 8) for intermittently stimulating muscles around the knee joint to facilitate a better walking gait, which may be separated from the knee brace 101 or formed integrally. The knee brace may be secured on the lower limb 10 using fastening means 105. In certain embodiments, the fastening means 105 may be any kind of connection including, but not limited to, Velcro fasteners, Ladder straps, hook and loop connections, buttons, magnetic connectors, zippers, or other similar devices.

The knee brace (the lower arm 101 and the upper arm 102) may be made of biocompatible materials as the knee exoskeleton device 100 is constantly in contact with patients when used. In particular, the material complies with biocompatibility tests of at least the International Organization for Standardization (ISO) 10993-1 (Evaluation and testing within a risk management process), the ISO 10993-5 (Test for in vitro cytotoxicity), and the ISO 10993-10 (Tests for irritation and skin sensitization). Therefore, the knee brace can be made of rigid and lightweight materials selected from the group consisting of, but not limited to, carbon fiber, carbon composite, light metal, or plastic. The weight of the knee exoskeleton device 100 can be less than 1 kg. In certain embodiments, the knee brace is padded with soft materials, e.g., cotton, leather, rubber, plastic, or silicone, to increase the level of comfort. Similar compliance between the soft materials and human tissues brings minimal harm to the user. Optionally, the knee exoskeleton device 100 complies with IPX2 Rainfall Drip Testing.

As the rotary motion generator 200 couples to the lower arm 101 and the upper arm 102 of the knee brace, the rotary motion generator 200 is pneumatically driven to cause a relative rotatory movement of the lower arm 101 with respect to the upper arm 102 about an axis 230 parallel to or coincide with a rotation axis of a joint for facilitating a movement of a limb, such as a knee flexion and a knee extension during walking. The knee exoskeleton device 100 provides an output torque for producing the relative rotatory movement of the lower arm 101 in a sagittal plane with a range of motion for moving the knee joint. Although the description provides a device for use on the lower limb at the knee joint, it is apparent that the present disclosure may also be applied to other joints for assisting the motion of the corresponding joint. Advantageously, the rotary motion generator 200 is capable of locking a joint position.

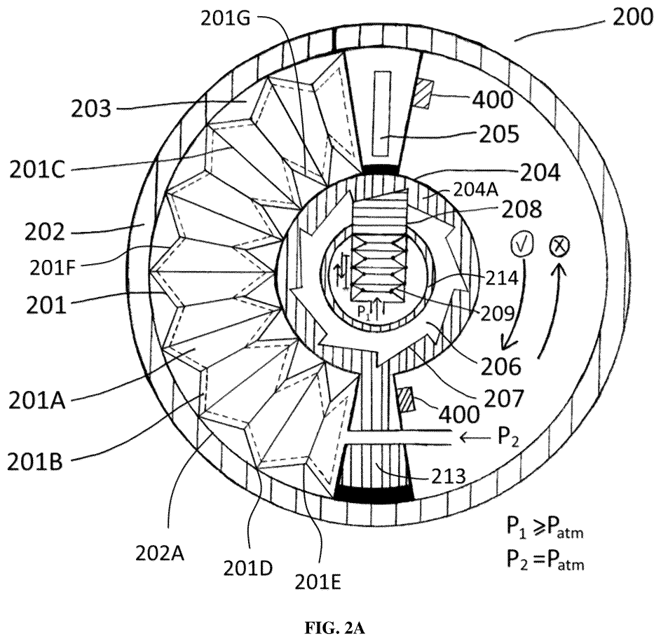

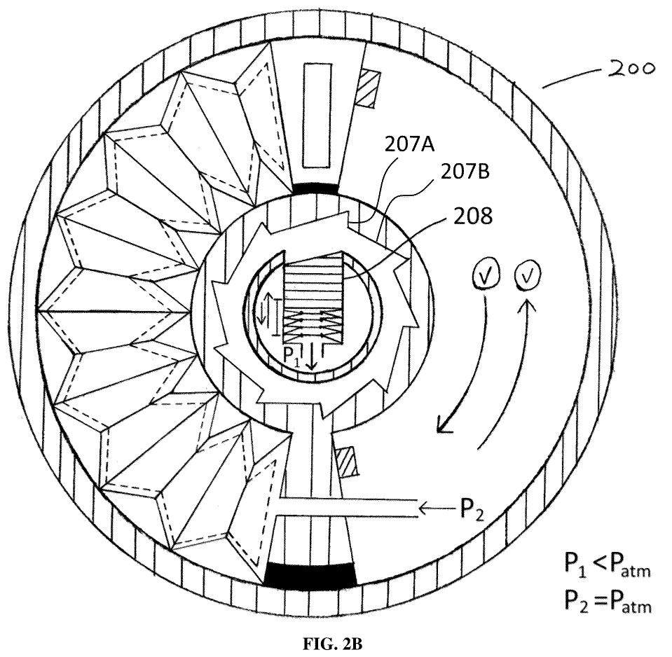

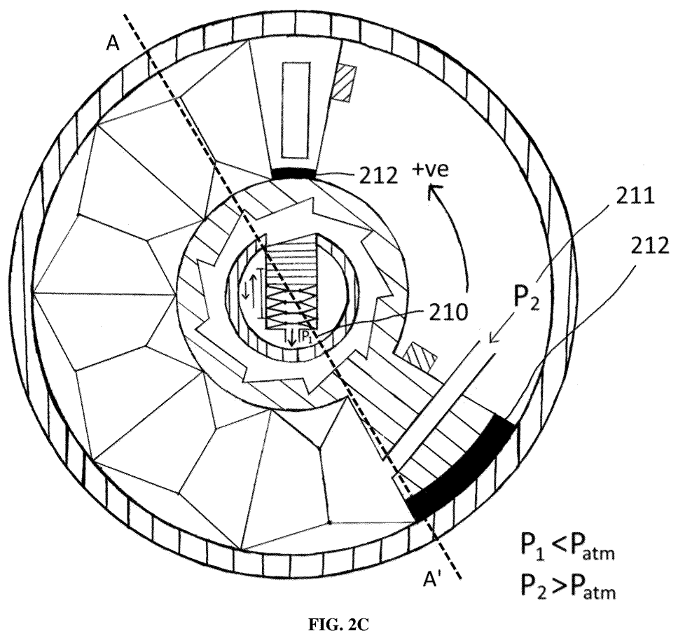

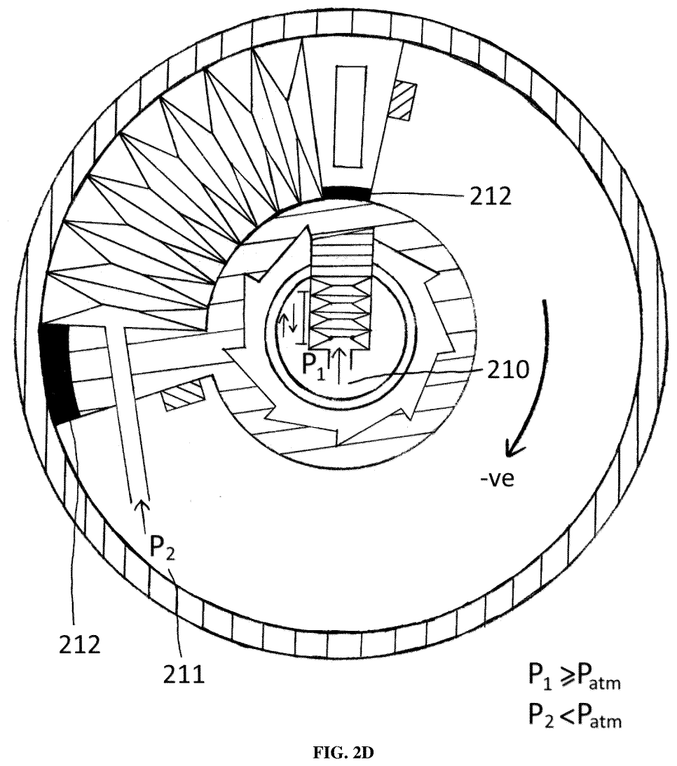

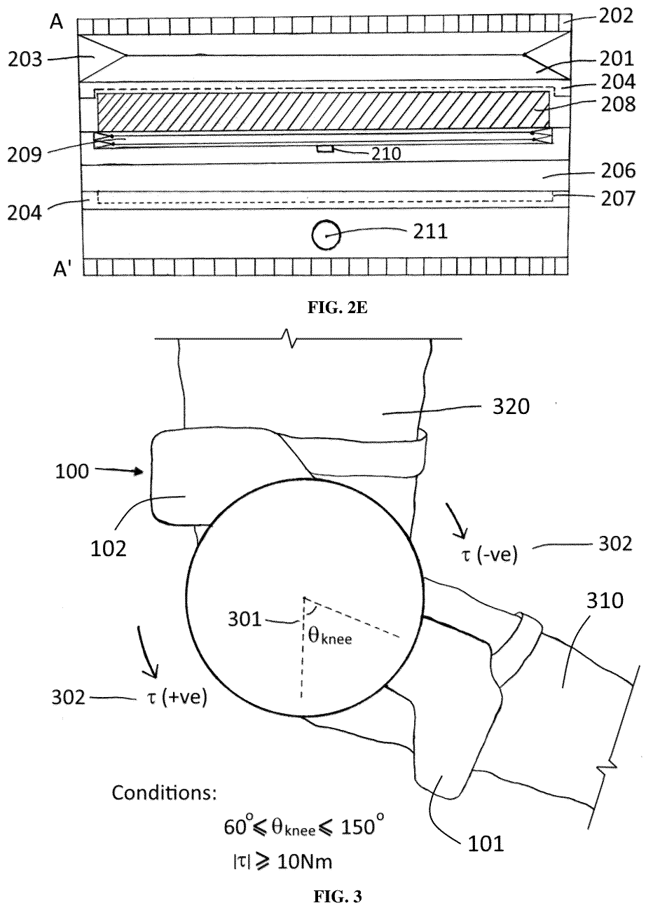

The first embodiment of the present disclosure provides a rotary motion generator 200 coupled to the lower arm 101 and the upper arm 102 for permitting rotation of the lower arm 101, as shown in FIG. 2A. The rotary motion generator 200 comprises an outer casing 202, a rotary shaft 204, a first elastomeric structure 201, and a radial extension 205 extended radially from the outer casing 202 to the rotary shaft 204. The outer casing 202 may be a hollowed cylinder shell defining an internal space 203 between an inner wall 202A of the outer casing 202 and the rotary shaft 204. The radial extension 205 is affixed to or formed as an integral part of the outer casing 202. The rotary shaft 204 is rotatable about the axis 230 to allow the lower arm 101 to move with respect to the upper arm 102. The rotary motion generator 200 may also be made of biocompatible materials complying with biocompatibility tests of ISO 10993-1 and ISO 10993-10.

The rotary motion generator 200 further comprises a movable lever 213, which is protruded from the outer periphery of the rotary shaft 204 perpendicularly for rotating the rotary shaft 204. The first elastomeric structure 201 may be suitably modified or configured for generating an output torque and produce the relative rotatory movement of the lower arm 101 with respect to the upper arm 102. In the illustrated embodiments, the first elastomeric structure 201 comprises a bellow tube structure, which is circumferentially arranged within the outer casing 202 at least partially occupying the internal space 203. It is apparent that the first elastomeric structure 201 may be presented in other positions within the outer casing 202 without departing from the scope and spirit of the present invention. The first elastomeric structure 201 comprises a first end fixed at the radial extension 205 connected to the outer casing 202, and a second end engaged with the moveable lever 213. When the first elastomeric structure 201 expands and elongates, the second end pushes the moveable lever 213 to rotate in an anticlockwise direction. When the first elastomeric structure 201 compresses and retracts, the second end allows the moveable lever 213 to rotate in a clockwise direction. Therefore, the first elastomeric structure 201 is pneumatically controlled for engaging the moveable lever 213.

In certain embodiments, the bellow tube structure of the first elastomeric structure 201 is formed by a plurality of outer convolutions 201F and a plurality of inner convolutions 201G, defined by alternating crests 201D and troughs 201E. The plurality of outer convolutions 201F and the plurality of inner convolutions 201G allow compression or expansion of the first elastomeric structure 201 based on a pressure change supplied to an undulated cavity 201A of the first elastomeric structure 201. The bellow tube structure may have substantially V-shaped convolutions, or U-shaped convolutions (not shown in the drawings). The first elastomeric structure 201 has a wall thickness 201B sufficient to sustain the expansion of the first elastomeric structure 201.

The bellow tube structure further comprises a structural mesh 201C connecting the plurality of outer convolutions 201F and the plurality of inner convolutions 201G for supporting the first elastomeric structure 201 and preventing an expansion of the first elastomeric structure 201 in an axial direction. The structural mesh 405 is formed using supporting lengthwise filaments, supporting widthwise filaments, or a patterned mesh selected from a net mesh or a double helical mesh. In one embodiment, the structural mesh 405 is impregnated within the first elastomeric structure 201 or mounted to a surface of the first elastomeric structure 201. The structural mesh 405 connects crests 201D of the plurality of outer convolutions 201F with the plurality of inner convolutions 201G, and troughs 201E of the plurality of outer convolutions 201F with the plurality of inner convolutions 201G. The structural mesh 405 can be made of any metals, for example, titanium alloy, stainless steel, or iron, that are hard to be broken by the fluid pressure inside the first elastomeric structure 201 due to pressurizing the undulated cavity 201A.

The rotary shaft 204 further comprises an inner housing 214, a hollowed body 204A, and a rotation locking mechanism 206. The inner housing 214 may be a circular tube concentrically arranged with respect to the rotary shaft 204 and within the hollowed body 204A. The inner housing 214 may be affixed to the outer casing 202, so the inner housing 214 is not rotated when the rotary shaft 204 rotates. The rotation locking mechanism 206 is provided to switch the rotary shaft 204 between a one-way rotation mode and a two-way rotation mode, which can be understood with reference to FIGS. 2A-2B. With the one-way rotation mode, the rotary motion generator 200 can lock the joint position to support the body weight of the user. In certain embodiments, the rotation locking mechanism 206 comprises at least a pawl 208 and a plurality of ratchet teeth 207, wherein the plurality of ratchet teeth 207 may be circumferentially arranged on an inner periphery of the rotary shaft 204. The pawl 208 is supported by the inner housing 214, which can restrict the bidirectional movement of the rotary shaft 204. In particular, the pawl 208 engages the plurality of ratchet teeth 207 to restrict the rotation of the rotary shaft 204 in an anticlockwise direction. In certain embodiments, the rotation locking mechanism 206 further comprises a second elastomeric structure 209 located inside the inner housing 214 to manipulate the movement of the pawl 208. The pawl 208 is projected out from the inner housing 214 by the second elastomeric structure 209 for locking the rotary shaft 204, or retracted for unlocking the rotary shaft 204. The second elastomeric structure 209 has a bellow tube structure that expands or retracts longitudinally to move the pawl 208 between an engaged position and a disengaged position. Therefore, the rotation locking mechanism 206 switches the rotary shaft 204 between the one-way rotation mode and the two-way rotation mode by pneumatically controlling the second elastomeric structure 209. It is apparent that the second elastomeric structure 209 may be presented in other positions within the inner housing 214 or outside the inner housing 214 without departing from the scope and spirit of the present invention.

In certain embodiments, the shapes of the pawl 208 and any one of the plurality ratchet teeth 207 are matched. Each of the plurality of ratchet teeth 207 comprises a blocking portion 207A and a sloped portion 207B. When the pawl 208 is at the engaged position, the pawl 208 engages the plurality of ratchet teeth 207 by abutting the pawl 208 against any one of the plurality ratchet teeth 207. The blocking portion 207A prevents the rotary shaft 204 from rotating about the axis in an anticlockwise direction. However, as the sloped portion 207B has a shape that matches the pawl 208, the pawl 208 is slidable along the sloped portion 207B so that the rotary shaft 204 can rotate in the clockwise direction to an adjacent ratchet tooth. Therefore, when the pawl 208 is at the engaged position, the pawl 208 is projected from the inner housing 214 by the second elastomeric structure 209 to pivot between the inner housing 214 and the plurality of ratchet teeth 207 for restricting the rotary shaft 204 from rotating about the axis 230 in the anticlockwise direction. Referring to FIG. 2B, when the pawl 208 is at the disengaged position, the pawl 208 disengages the plurality of ratchet teeth 207 by retracting into or partially into the inner housing 214 by the second elastomeric structure 209. The rotary shaft 204 is allowed to rotate bidirectionally about the axis.

The contact surfaces between the outer casing 202 and the rotary shaft 204, between the first elastomeric structure 201 and the rotary shaft 204, and between the outer casing 202 and the structural mesh 201C may be lubricated with a lubricant 212. The lubricant 212 can reduce the induced friction during rotation. In exemplary embodiments, the lubricant 212 may be grease, oil, or the like. The optimal coefficient of friction (COF) between surfaces can be no greater than 0.1, which is the less the better. In certain embodiments, the rotating shaft 204 can be constructed to be not contacting with the first elastomeric structure 201 or the outer casing 202. The lubricant 212 can be applied to the pawl 208 and the plurality of ratchet teeth 207 of the rotation locking mechanism 206 to allow a smooth transition between the one-way rotation mode and the two-way rotation mode of the rotating shaft 204 by controlling the second elastomeric structure 209.

Referring to FIGS. 2A-2B, the second elastomeric structure 209 elongates or retracts depending on the first pressure (P₁). When a negative pressure (less than the atmospheric pressure) is applied to the second elastomeric structure 209 with a second fluid supply through a second tube 210, the pawl 208 is retreated inwardly to disengage the plurality of ratchet teeth 207. The rotary shaft 204 is unlocked for rotating. When a positive pressure (more than or equal to the atmospheric pressure) is applied to the second elastomeric structure 209 with a second fluid supply through a second tube 210, the pawl 208 stays at the initial position to engage the plurality of ratchet teeth 207. The rotary shaft 204 is locked from rotating in an anticlockwise direction, but can still rotate in a clockwise direction.

Referring to FIGS. 2C-2D, the first elastomeric structure 201 elongates or retracts depending on the second pressure (P₂). When a negative pressure (less than the atmospheric pressure) is applied to the first elastomeric structure 201 with a first fluid supply through a first tube 211, the first elastomeric structure 201 retracts and the moveable lever 213 rotates in a clockwise direction. When a positive pressure (more than or equal to the atmospheric pressure) is applied to the first elastomeric structure 201 with a first fluid supply through the first tube 211, the first elastomeric structure 201 elongates and the moveable lever 213 rotates in an anticlockwise direction if the rotation locking mechanism 206 is disengaged.

With reference to FIG. 5, the first fluid supply and the second fluid supply are provided to the first and second elastomeric structures 201, 209 from a control device 704 using a tube 702, such as, a rubber tube, a PE tube, or a PVC tube. The tube 702 may separately connect the first tube 211 and the second tube 210, so the control device 704 can adjust the pressure of the first and second elastomeric structures 201, 209. The control box 704 may be worn on the waist of the users by any means, for example, a belt or a girdle. In certain embodiments, the control device 704 further comprises a pressure source and a solenoid valve. The pressure source may supply pressurized fluid at the first pressure (P₁) to the second elastomeric structure 209 and the second pressure (P₂) to the first elastomeric structure 201. The solenoid valve is configured to control the first pressure and the second pressure supplied to the first and second elastomeric structures 201, 209 of the rotary motion generator 200. The pressure source can be a pump, a cylinder, a compressor, or any disposable or non-disposable compressed medium including, for example, a carbon dioxide bottle, oxygen tank, compressed nitrogen, or compressed air. The fluid supplied to the first and second elastomeric structures 201, 209 can be a gas or a liquid, and can be recycled or disposable. Specific examples of gasses include compressed carbon dioxide, air, or nitrogen. The control device 704 may also include a vacuum pump, which is used to create a vacuum by removing fluid from the first and second elastomeric structures 201, 209. The vacuum pump may be, for example, a plunger or a syringe. As the knee exoskeleton device 100 is pneumatically driven by a fluid, this allows the rotary motion generator 200 to be lightweight and compliant with human tissue.

FIG. 2E shows the cross-section view of the rotary motion generator 200 along the axis A-A′ of FIG. 2C with details of the first elastomeric structure 201 and the second elastomeric structure 209. The first tube 211 and the second tube 210 may have different diameters for pressurizing the first and second elastomeric structures 201, 209.

FIG. 3 shows the actuation of the knee exoskeleton device 100. The upper arm 102 is in contact with the thigh 320 of the user, and the lower arm 101 is in contact with the shank 310 of the user. Preferably, the upper arm 102 and the lower arm 101 both have a curved surface to conform with the shape of the thigh 320 and the shank 310. With the fastening means 105, the rotary motion generator 200 can be secured next to the knee joint when the knee exoskeleton device 100 is worn, such that the lower arm 101 can be rotated with respect to the upper arm 102 about an axis parallel to or coincide with a rotation axis of the knee joint for facilitating the movement of the shank 310.

The rotary motion generator 200 provides a range-of-motion 301 and an output torque 302 for a movement of the knee in a sagittal plane according to an embodiment of the present disclosure. The range-of-motion 301 and the output torque 302 depend on the statistical characteristics of the knee of a typical user. The maximum range-of-motion of the knee joint was found to be between 130° and 150°, based on the scientific journal article by Roach and Miles (Table 1, “Normal Hip and Knee Active Range of Motion: The Relationship to Age”, 1991). However, the normal range-of-motion 301 of the knee joint during walking was found to be approximately 60°, based on the scientific journal article by Brinkmann and Perry (Table 1, “Rate and Range of Knee Motion During Ambulation in Healthy and Arthritic Subjects”, 1985). Therefore, the range-of-motion 301 of 60° is applied for the purpose of the rotary motion generator 200 to accommodate with active movement of the knee. On the other hand, the output torque 302 required to move the knee joint of a paretic leg after stroke was shown more than 10 N-m, and up to 50 N-m for a non-paretic leg, based on the scientific journal article by Chaparro-Rico, Cafolla, Tortola and Galardi (Table 3, “Assessing Stiffness, Joint Torque and ROM for Paretic and Non-Paretic Lower Limbs during the Subacute Phase of Stroke Using Lokomat Tools, 2020). As a result, the rotary motion generator 200 is configured to generate an output torque 302 in a range of 10 N-m to 50 N-m and maintain a range-of-motion 301 of the knee joint from 60° to 150°. Fluid pressure supplied to the first elastomeric structure 201 can be adjusted such that the range-of-motion 301 and the output torque 302 can be either increased or decreased depending on the ongoing walking condition of the user. However, it is limited to a maximum pressure of 600 kPa compared with the atmospheric pressure. The value is taken with reference to the maximum positive pressure of a typical portable fluid pump.

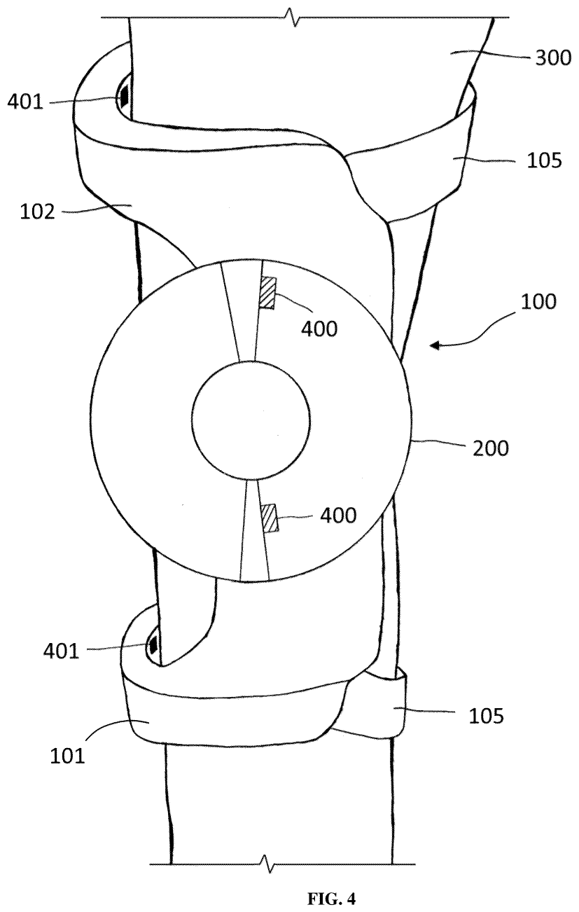

Referring to FIG. 4, the knee exoskeleton device 100 may comprise a sensor system that includes one or more sensors integrated into the knee exoskeleton device 100 to provide feedback about the movement of the joint and determine an ongoing gait event. In certain embodiments, the sensor system comprises one or more motion sensors 400 disposed within the rotary motion generator 200 to monitor an angular displacement, a linear displacement, a velocity, and an acceleration of the rotary shaft 204. In the illustrated embodiments of FIG. 2A, a first motion sensor is attached to the radial extension 205, and a second motion sensor is attached to the movable lever 213. The one or more motion sensors 400 are arranged for analyzing a spatial relationship between the thigh 320 and the shank 310, thereby the knee joint configuration (flexion or extension), the knee joint range-of-motion, the knee joint trajectories during walking, and the knee joint spasticity can be determined. In certain embodiments, the one or more motion sensors 400 can also be installed to the knee brace, the fastening means 105, or otherwise attached to the user at the waist, the thigh 320, the shank 310, or the ankle without departing from the scope and spirit of the present disclosure. The readings obtained from the one or more motion sensors 400 can be supportive for indicating the ongoing gait events 600, and the intention of the users by moving the knee joint when walking with the knee exoskeleton device 100. The motion sensor 400 may include, but is not limited to, an inertial measurement unit (IMU), an angle encoder, a potentiometer, a strain gauge, a gyroscope, or a flex sensor.

The sensor system further comprises one or more force sensors 401 for measuring the contact force exerted on the knee exoskeleton device 100 at positions of the thigh 320 and the shank 310. In case the contact force at either one position is greater than that at another position, it indicates the intention of the users to move the knee joint. The one or more force sensors 401 can be a thin film force sensitive resistor (FSR), a force transducer, a strain gauge, a pressure sensor, or the like. In some embodiments, the one or more force sensors 401 can also be installed on the foot of the users, for example, the heel or the toe, for measuring the stepping force during walking, such that the readings from the one or more force sensors 401 can be indicative to the ongoing gait events 600 when the user is walking with the knee exoskeleton device 100.

A second embodiment of the present disclosure provides an electrical stimulation system 700 comprising a plurality of electrodes 701 for applying electrical current pulses 508 to the muscular tissue regions to facilitate a better gait, as illustrated in FIG. 5. The electrical stimulation system 700 can stimulate the lower limb during walking and help a patient with less or even no residual ability to walk to use it for relearning walking. The plurality of electrodes 701 are arranged to contact a plurality of regions of the thigh 320 and the shank 310. In certain embodiments, two of the plurality of electrodes 701 are arranged to stimulate the quadriceps muscle group, including but not limited to the vastus intermedius, vastus medialis, vastus lateralis, or rectus femoris. Another two of the plurality of electrodes 701 are arranged to stimulate the hamstring muscle group, including but not limited to the long head of the biceps femoris, short head of the biceps femoris, semitendinosus, or semimembranosus. Electric wires 703 may connect the plurality of electrodes 701 to the control device 704. The control device 704 further comprises a microcontroller. In certain embodiments, the microcontroller may be implemented using one or more of: CPU, MCU, controllers, logic circuits, Arduino, Raspberry Pi chip, ATmega, Intel 8051, other digital signal processors (DSP), application-specific integrated circuit (ASIC), Field-Programmable Gate Array (FPGA), or any other digital or analog circuitry configured to interpret and/or to execute program instructions and/or to process information and/or data. The microcontroller may receive force signals, displacement signals, velocity signals, acceleration signals, and fluid pressure signals as input signals.

In certain embodiments, the control device 704 further comprises a communication device 705, for example, a Bluetooth or Wi-Fi module, for establishing a wireless communication between a computer 706 and the knee exoskeleton device 100. Readings from the one or more motion sensors 400 and the one or more force sensors 401 can be transmitted wirelessly from the control box 704 to the computer 706 through the communication device 705 and displayed on the screen. The computer 706 can issue commands wirelessly to the knee exoskeleton device 100 through the communication device 705 as well, such that the microcontroller can control the fluid pressure by regulating the pressure source, pressure regulator, or control valve. The computer 706 can be, but is not limited to, a tablet, a desktop, or a laptop.

The communication device 705 may include one or more of: a modem, a Network Interface Card (NIC), an integrated network interface, an NFC transceiver, a ZigBee transceiver, a Wi-Fi transceiver, a Bluetooth® transceiver, a radio frequency transceiver, an optical port, an infrared port, a USB connection, or other wired or wireless communication interfaces. The transceiver may be implemented by one or more devices (integrated transmitter(s) and receiver(s), separate transmitter(s) and receiver(s), etc.). The communication link(s) may be wired or wireless for communicating commands, instructions, information, and/or data.

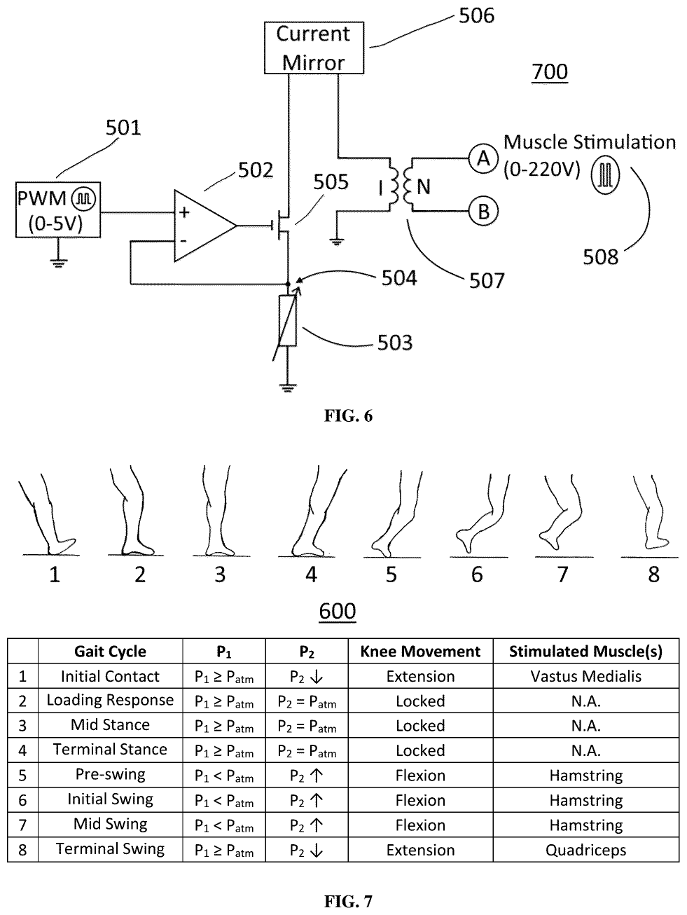

FIG. 6 is a schematic diagram of the electrical stimulation system 700. A pulse-width modulation (PWM) signal 501 for controlling an intensity of muscle stimulation is generated by a PWM generator, such as a timer (for example, IC555), a signal generator, or a computer program (for example, LabView, MATLAB, Python, and other application programs). The amplitude of the PWM signal can be adjusted between 0V and 5V. The PWM signal 501 is coupled to an operational amplifier 502 to clamp the voltage across a resistor 503 at node 504, such that a triode 505, for example, a relay, a solid-state relay, a transistor, a MOSFET, or other switching elements, can be used to act as a switching device for allowing a constant current to pass through. A current mirror circuit 506, for example, Widlar current source, Wilson current mirror, or Cascode current mirror, can be used to copy the current from the triode 505 to the primary coil of a transformer 507. The transformer 507 has a primary coil connected to the current mirror circuit 506 and a secondary coil connected to the plurality of electrodes 701. Therefore, electrical current pulses 508 can be applied to the muscles of the user, for example, the hamstring muscle group, the quadriceps muscle group, or specifically the vastus medialis muscle, for stimulating their contraction. The turn ratio between the primary coil and the secondary coil may be at least 1:10 or greater. The amplitude of the electrical current pulses 508 can be adjusted to a range between 0 V to 220 V depending on the input PWM signal 501. The electrical stimulation system 700 is configured to generate electrical current pulses of a predetermined current amplitude independent of a voltage level and a human skin resistance across any two of the plurality of electrodes 701. The electrical current pulses 508 may have a square waveform or a triangular waveform.

One of the advantages of the present invention is that the control for the rotary motion generator 200 and the electrical stimulation system 700 of the knee exoskeleton device 100 operate adaptively and intermittently based on an ongoing gait event 600. A normal gait pattern of humans is shown in FIG. 7, which describes a gait cycle of a person when walking. The gait cycle comprises a plurality of gait phases: (1) initial contact; (2) loading response; (3) mid stance; (4) terminal stance; (5) pre-swing; (6) initial swing; (7) mid swing; and (8) terminal swing. In certain embodiments, the rotary motion generator 200 is configured to assist with knee extension, knee flexion, or to lock a joint position for supporting the body weight. The electrical stimulation system 700 is configured to stimulate the hamstring muscle group, the quadriceps muscle group, or specifically the vastus medialis muscle.

The electrical stimulation system 700 is configured to stimulate the hamstring muscle group and the rotary motion generator 200 is configured to assist with the knee flexion in the gait phases of pre-swing, initial swing, and mid-swing. In the following gait phase of terminal swing, the electrical stimulation system 700 is configured to stimulate the quadriceps muscle group, and the rotary motion generator 200 is configured to assist with the knee extension. At initial contact, knee extension can be further strengthened by stimulating the vastus medialis muscle, and the rotary motion generator 200 is configured to assist the knee extension. For the subsequent gait phases of loading response, mid stance, and terminal stance, the electrical stimulation system is stopped and no muscles would be stimulated. There is also no pressure applied to the first elastomeric structure 201. The rotary motion generator 200 can therefore lock the joint position and maintain the knee extension by the rotation locking mechanism 206 of the rotary motion generator 200 for supporting the body weight. With the knee exoskeleton device 100 of the present disclosure, the knee can be properly locked for supporting the body weight of the patients in different gait phases, which is a novel way to help a patient with walking difficulties. Alternatively, the second elastomeric structure 209 can be supplied with a negative pressure from pre-swing to mid-swing to retreat the pawl 208 to the disengaged position such that the first elastomeric structure 201 can control the flexion of the knee upon positive pressurization. The second elastomeric structure 209 can be pressurized again to return the pawl 208 to the engaged position during knee extension, and therefore moving towards the flexion direction of the rotary shaft 204 would be limited. The first elastomeric structure 201 can be deflated, i.e., applied with a negative pressure, at the same time to extend the knee.

FIG. 8 shows a block diagram of an exemplary knee exoskeleton device 100 in accordance with certain embodiments of the present disclosure. A control device 704 is provided for controlling the rotary motion generator 200 and the plurality of electrodes 701 of the electrical stimulation system 700. The control device 704 may comprise one or more components selected from the group consisting of the microcontroller, the pressure source, the pressure regulator, the solenoid valve, a communication device 705, and a battery receptacle for receiving a power source for powering the knee exoskeleton device 100. The battery receptacle may be arranged to receive one or more batteries, such as but not limited to one or more replaceable lithium batteries. Input control signals may be provided to the control device 704 using an input device or via the communication device 705. The input device may include one or more of: keyboard, mouse, stylus, image scanner (e.g., barcode identifier and QR code), microphone, touch-sensitive screen, image/video input device (e.g., camera device), biometric data input device (e.g., fingerprint detector, facial detector), and other sensors. A person skilled in the art would appreciate that the control device 704 described above is merely exemplary and that the control device 704 may have different configurations (e.g., additional components, fewer components, etc.).

Although the illustrated embodiment provides that the rotary motion generator 200 is used in a knee exoskeleton device 100 for facilitating a movement of a lower limb, it is apparent that the rotary motion generator 200 may also be used in any robotic devices without departing from the scope and spirit of the present disclosure. When the rotary motion generator 200 is used in other robotic devices, the rotary motion generator 200 is pneumatically driven to cause the rotation of a first element with respect to a second element about an axis to facilitate the movement of a joint.

It will also be appreciated that where the systems of the present disclosure are either wholly implemented by a computing system or partly implemented by computing systems then any appropriate computing system architecture may be utilized. This will include stand-alone computers, network computers, and dedicated or non-dedicated hardware devices.

This illustrates the fundamental structure of the knee exoskeleton device 100 in accordance with the present disclosure. It will be apparent that variants of the above-disclosed and other features and functions, or alternatives thereof, may be combined into many other different kinds of assistive devices. The present embodiment is, therefore, to be considered in all respects as illustrative and not restrictive. The scope of the disclosure is indicated by the appended claims rather than by the preceding description, and all changes that come within the meaning and range of equivalency of the claims are therefore intended to be embraced therein.

What is claimed is:

1. A rotary motion generator for use in a robotic device, the rotary motion generator being pneumatically driven to cause a relative rotatory movement of a lower arm with respect to an upper arm about an axis parallel to or coincide with a rotation axis of a joint for assisting a joint extension, a joint flexion and capable of locking a joint position, the rotary motion generator comprising:
a rotary shaft switchable between a one-way rotation mode and a two-way rotation mode in a sagittal plane, and locking the joint position by the one-way rotation mode;
a moveable lever protruding from an outer periphery of the rotary shaft perpendicularly for rotating the rotary shaft; and
a first elastomeric structure pneumatically controlled for engaging the moveable lever to generate an output torque and produce the relative rotatory movement of the lower arm,
wherein:
the rotary shaft further comprises at least a pawl, a second elastomeric structure, and a rotation locking mechanism; and
the rotation locking mechanism switches the rotary shaft between the one-way rotation mode and the two-way rotation mode by pneumatically controlling the second elastomeric structure to project or retract the pawl.

2. The rotary motion generator of claim 1, wherein the rotation locking mechanism further comprises a plurality of ratchet teeth, wherein the pawl restricts a bidirectional movement of the rotary shaft when engages with the plurality of ratchet teeth.

3. The rotary motion generator of claim 2, wherein the plurality of ratchet teeth are circumferentially arranged on an inner periphery of the rotary shaft.

4. The rotary motion generator of claim 3, wherein the second elastomeric structure has a bellow tube structure that moves the pawl between an engaged position and a disengaged position, wherein:
the rotary shaft is prevented from rotating about the axis in an anticlockwise direction when the pawl is in the engaged position; and
the rotary shaft is allowed to rotate bidirectionally about the axis when the pawl is in the disengaged position.

5. The rotary motion generator of claim 4, wherein the plurality of ratchet teeth comprises a blocking portion and a sloped portion, wherein:
the pawl engages the plurality of ratchet teeth by abutting the pawl against any one of the plurality ratchet teeth in the engaged position; and
the blocking portion limits the rotary shaft from rotating in the anticlockwise direction.

6. The rotary motion generator of claim 5, wherein when the pawl is in the engaged position, the pawl is slidable along the sloped portion so that the rotary shaft can rotate in the clockwise direction to an adjacent ratchet tooth.

7. The rotary motion generator of claim 4, wherein the rotary shaft further comprises an inner housing and a hollowed body, wherein:
the inner housing is concentrically arranged with respect to the rotary shaft and within the hollowed body, and is not rotated when the rotary shaft rotates; and
when the pawl is at the engaged position, the pawl is projected from the inner housing by the second elastomeric structure to pivot between the inner housing and the plurality of ratchet teeth for restricting the rotary shaft from rotating about the axis in the anticlockwise direction.

8. The rotary motion generator of claim 7, wherein when the pawl is at the disengaged position, the pawl is retracted into or partially into the inner housing by the second elastomeric structure to allow the bidirectional movement of the rotary shaft.

9. The rotary motion generator of claim 1 further comprising an outer casing and a radial extension extended radially from the outer casing to the rotary shaft, wherein the first elastomeric structure is circumferentially arranged within the outer casing, and comprises a first end fixed at the radial extension and a second end engaged with the moveable lever.

10. The rotary motion generator of claim 9, wherein the radial extension is affixed to or formed as an integral part of the outer casing.

11. The rotary motion generator of claim 9, wherein the outer casing is a hollowed cylinder shell defining an internal space between an inner wall of the outer casing and the rotary shaft, wherein the first elastomeric structure is circumferentially arranged within the outer casing at least partially occupying the internal space.

12. The rotary motion generator of claim 1, wherein the first elastomeric structure comprises a bellow tube structure formed by a plurality of outer convolutions and a plurality of inner convolutions, wherein:
the plurality of outer convolutions and the plurality of outer convolutions are defined by alternating crests and troughs; and
the plurality of outer convolutions and the plurality of inner convolutions allow compression or expansion of the first elastomeric structure based on a pressure change supplied to an undulated cavity of the first elastomeric structure.

13. The rotary motion generator of claim 12, wherein the bellow tube structure has V-shaped convolutions or U-shaped convolutions.

14. The rotary motion generator of claim 12, wherein the first elastomeric structure comprises a structural mesh connecting the plurality of outer convolutions and the plurality of inner convolutions for supporting the first elastomeric structure and preventing an expansion of the first elastomeric structure in an axial direction.

15. The rotary motion generator of claim 14, wherein the structural mesh is formed using supporting lengthwise filaments, supporting widthwise filaments, or a patterned mesh selected from a net mesh or a double helical mesh.

16. The rotary motion generator of claim 14, wherein the structural mesh is impregnated within the first elastomeric structure or mounted to a surface of the first elastomeric structure.

17. The rotary motion generator of claim 1, wherein the output torque generated is in a range of 10 N-m to 50 N-m; and the rotary motion generator is configured to maintain a range-of-motion of the joint from 60° to 150°.

18. A knee exoskeleton device, comprising:
a knee brace having a lower arm and an upper arm;
a rotary motion generator having a rotary shaft, the rotary motion generator coupled to the lower arm and the upper arm for permitting rotation of the lower arm with respect to the upper arm about an axis parallel to or coincide with a rotation axis of a joint for assisting a joint extension, a joint flexion and capable of locking a joint position; and
an electrical stimulation system comprising a plurality of electrodes for intermittently stimulating muscles in a lower limb to facilitate a walking gait,
wherein:
the plurality of electrodes are arranged for contacting a plurality of regions of a thigh and a shank to apply electrical current pulses to hamstring muscle group and quadriceps muscle group; and
the rotary motion generator and the electrical stimulation system operate adaptively and intermittently based on an ongoing gait event.

19. The knee exoskeleton device of claim 18 further comprising a sensor system for determining the ongoing gait event, the sensor system comprising:
one or more motion sensors disposed within the rotary motion generator to monitor an angular displacement and a linear displacement a velocity, and an acceleration of the rotary shaft; and
one or more force sensors for measuring the contact force exerted on the knee exoskeleton device at positions of the thigh and the shank.

20. The knee exoskeleton device of claim 18, wherein:
the rotary motion generator comprises:
an outer casing and a radial extension extended radially from the outer casing to the rotary shaft; and
a moveable lever protruding from an outer periphery of the rotary shaft perpendicularly for rotating the rotary shaft; and
the one or more motion sensors comprises a first motion sensor attached to the radial extension, and a second motion sensor attached to the movable lever, thereby the one or more motion sensors are arranged for analyzing a spatial relationship between the thigh and the shank.

21. The knee exoskeleton device of claim 18, wherein the electrical stimulation system is configured to generate electrical current pulses of a predetermined current amplitude independent of a voltage level and a human skin resistance across any two of the plurality of electrodes.

22. The knee exoskeleton device of claim 21, wherein the electrical stimulation system comprises a pulse-width modulation (PWM) generator, an operational amplifier, a triode, a current mirror circuit, and a transformer having a primary coil connected to the current mirror circuit and a secondary coil connected to the plurality of electrodes.

23. The knee exoskeleton device of claim 22, wherein the electrical current pulses have a square waveform or a triangular waveform.

24. The knee exoskeleton device of claim 18, wherein:
the electrical stimulation system is configured to stimulate the hamstring muscle group, and the rotary motion generator is configured to assist with the knee flexion during pre-swing, initial swing, and mid-swing;
the electrical stimulation system is configured to stimulate the quadriceps muscle group, and the rotary motion generator is configured to assist with the knee extension during terminal swing;
the electrical stimulation system is configured to stimulate a vastus medialis muscle, and the rotary motion generator is configured to assist with the knee extension during initial contact; and
the electrical stimulation system is stopped, and the rotary motion generator is configured to lock the joint position during loading response, mid stance, and terminal stance.
